(19) United States
(12) Patent Application Publication
(10) Pub. No.: US 2008/0306167 A1
(43) Pub. Date: Dec. 11, 2008

(54) Metal Pigment Composition

(76) Inventor: Fabrice Morvan, Oloron Sainte Marie (FR)

Correspondence Address: CROWELL & MORING LLP;INTELLECTUAL PROPERTY GROUP, P.O. BOX 14300, WASHINGTON, DC, 20044-4300, US

(21) Appl. No.: 11/793,711

(22) PCT Filed: Dec. 21, 2005
(86) PCT No.: PCT/FR05/03224

§ 371 Date: Apr. 18, 2008

(30) Foreign Application Priority Data
Dec. 22, 2004 (FR) 0413727

(51) Int. Cl. A61K 8/19; C09B 67/10; A61Q 99/00; C09D 11/00

(52) U.S. Cl. 514/769; 106/504; 106/404; 106/403; 106/31.86

(57) ABSTRACT

The present invention relates to a composition formed of metal pigments and to a process for their preparation.

The composition is composed of metal particles FP and a liquid, the liquid being an ester of a fatty acid R′-COOR in which R′ is a saturated or unsaturated aliphatic group having from 9 to 20 carbon atoms and R is an alkyl having from 1 to 8 carbon atoms. It is obtained by a process which consists in introducing starting particles of the metal (SP) into a liquid vehicle and in subjecting the mixture thus obtained to milling, said liquid vehicle being said fatty acid ester.

Uses: preparation of paints, of inks, of plastics or of cosmetic compositions.

The present invention relates to a composition formed of metal pigments, in particular of aluminum pigments, for the preparation of metallic paints.

### BACKGROUND OF THE INVENTION

The technique conventionally used industrially for the preparation of compositions formed of metal pigments intended for the preparation of metallic paints consists in introducing the metal in the form of particles into white spirit and in subjecting the suspension to milling in the presence of a lubricating agent of the fatty acid type. This technique is described in particular in U.S. Pat. No. b 2,002,891. This technique makes it possible to obtain pigments having good properties. However, it exhibits a major disadvantage due to the high volatility and the toxicity of white spirit, which additionally is not biodegradable.

EP-0 936 253 describes a process for the preparation of a composition formed of metal particles which consists in milling particles of the metal in a medium consisting of the esterification product of a vegetable oil and comprising a fatty acid as lubricant. The medium essentially comprises a mixture of esters of the various fatty acids of which the vegetable oil used as source of esters is composed. There are advantages to this technique in comparison with the use of white spirit since the fatty acid esters are neither volatile nor toxic and in addition are biodegradable. However, the use as vehicle for the milling of metal particles of a mixture of esters derived from a vegetable oil and of a fatty acid as lubricant has certain disadvantages. First, it promotes the agglomeration of the particles during the aging of the pigment paste in which they are present during the storage thereof. In point of fact, the use, in the manufacture of a metallic paint, of a composition formed of metallic particles having a higher degree of agglomeration of the metal particles brings about a deterioration in the “metallic” appearance of the paint and an increase in the density of the associated color. This is because the agglomeration of the metal particles reduces the reflecting surface developed by the pigments, which thus lose their dyeing strength: in a colored lacquering composition comprising said particles, said pigments consequently bring about a reduced deterioration in the color of the lacquer comprising them. Moreover, the presence of the fatty acid in addition to the fatty acid esters in the composition formed of particles limits the effectiveness of the milling by providing an additional lubricating role. In addition, after the milling, the recovery of the milling solvent vehicle for the purpose of the recycling thereof requires a distillation.

The aim of the present invention is to provide novel compositions formed of metal pigments which do not exhibit the disadvantages of the compositions of the prior art as well as a process for their manufacture.

### SUMMARY OF THE INVENTION

The process according to the invention for the preparation of a composition formed of metal pigments consists in introducing starting particles of the metal (SP) into a liquid vehicle and in subjecting the mixture thus obtained to milling. It is characterized in that the liquid vehicle is composed of an ester of a fatty acid R′-COOR in which R′ is a saturated or unsaturated aliphatic group having from 9 to 20 carbon atoms and R is an alkyl having from 1 to 8 carbon atoms.

### DETAILED DESCRIPTION OF THE PREFERRED EMBODIMENTS

Preference is given, among the esters R′-COOR, to those in which R′ is a saturated CₙH₂ₙ₊₁ group, n being between 9 and 20, and more particularly those in which 9≦n≦17. Mention may be made, by way of example, of methyl caprate, methyl laurate, methyl myristate, methyl palmitate and methyl stearate.

The process can be employed for the preparation of compositions formed of metal pigments in which the metal is chosen from aluminum, copper, zinc, tin, gold, silver, alloys of these metals, and also stainless steels and bronze. It is particularly advantageous in the preparation of compositions formed of aluminum pigments which are used in particular in the preparation of metallic paints.

The metal particles SP can be in the form of any powder composed of essentially spherical or nonspherical particles or of small rods. They can also be formed by thin sheets, such as aluminum sheets. In the case of aluminum, use may in particular be made of powders composed of particles, the content of aluminum metal of which is at least 99% by weight, having a mean dimension of 0.1 to 500 μm. Mention may be made, by way of example, of the aluminum particles sold by Toyal Europe SA, Toyal America Inc. or Toyo Aluminium K.K. under the names “grade 406S”, “grade 409S” and “grade 432”.

Preferably, the mixture subjected to milling comprises from 1 to 10 kg of liquid vehicle per 1 kg of SP particles.

The aim of the milling is to plastically deform the starting metal particles SP which are converted, from a more or less spherical form or from a film form, into FP particles which have a form factor (representing the ratio of the mean thickness to the mean transverse diameter) of between 1/5 and 1/1000. Such particles can be provided in the form of glitter, of disks or of flakes which are more or less uniform. The shape and the dimension of the FP particles depends on the effectiveness of the milling. A less effective milling gives uniform FP particles of larger dimensions. If the milling is more effective, possibly under conditions of overmilling, the particles being formed are broken and give FP final particles of smaller dimensions which are possibly less uniform.

The duration of the milling is generally between 1 h and 20 h. It is adjusted according to the energy developed by the milling device, the nature of the SP particles and the result desired. The choice of the milling time is within the scope of a person skilled in the art.

At the end of the milling stage, the content of acid ester is adjusted to a value of between 30 and 90% by weight in order to form a composition composed of metal particles FP and the fatty acid ester which can be used directly for the various applications envisaged and which can be stored until it is used. The adjusting can be carried out by subjecting the mixture obtained after milling to the action of a filter press, in order to remove the excess liquid vehicle, or by adding fatty acid ester by mixing, if the content is inadequate.

Another subject matter of the invention is a composition formed of metal pigments which is composed of metal particles FP and a liquid, characterized in that the liquid is an ester of a fatty acid R′-COOR in which R′ is a saturated or unsaturated aliphatic group having from 9 to 20 carbon atoms and R is an alkyl having from 1 to 8 carbon atoms.

The compositions comprising an ester for which the R substituent is a methyl group and the R′ substituent is a saturated alkyl group having from 9 to 17 carbon atoms are particularly preferred.

The ester content of a composition according to the invention is preferably between 30 and 90% by weight.

The metal particles FP are anisotropic particles having mean dimensions of less than or equal to 500 μm and a form factor of between 1/5 and 1/1000. Preferably, the FP particles are of the glitter type with a mean transverse diameter of less than or equal to 500 μm and a mean thickness of less than or equal to 3 μm.

A composition formed of metal pigments according to the present invention can be used in various technical fields. For example, it can be used for the formulation of a metallic paint intended in particular for the automobile industry or for the formulation of industrial paints. A composition formed of metal pigments according to the invention can additionally be used for the formulation of a printing ink or of a plastic with a metallic appearance. In addition, a composition formed of pigments according to the invention can be used for the preparation of a cosmetic composition with a metallic appearance.

When a pigment composition according to the invention is used for the preparation of a metallic paint, the shape and the dimension of the FP particles has an influence on the appearance of the final paint. Generally, everything else otherwise being equal:
an increase in the mean transverse diameter of the particles brings about an increase in the gloss of a paint in which the particles are incorporated;
a decrease in the mean transverse diameter brings about a higher covering power which results more in a deterioration in the color associated with the paint;
the increase in the uniformity of the particles increases the gloss of the paint comprising them, whereas low uniformity gives dull paints;
an increase in the proportion of the particles promotes the metallic appearance of the paint.

The present invention is described in more detail with the help of the following examples, to which it is not, however, limited.

The products used are as follows:
conventional nonspherical aluminum powder, hereinafter denoted by Al−05 (d₅₀=5 μm, Al>99.7%);
conventional nonspherical aluminum powder, hereinafter denoted by Al−10 (d₅₀=10 μm, Al>99.7%);
methyl laurate, sold under the name Radia 7118 by Oleon;
colorless nitrocellulose lacquer, sold by Nouvion under the name NS813;
blue nitrocellulose lacquer, obtained by mixing nitrocellulose lacquer NS813 and phthalocyanine blue;
mixture of fatty acids comprising a high content of oleic acid (90%), sold by Oleon under the name Radiacid 294.

In the various examples,
the milling is carried out in a roll mill having a diameter of 50 mm and an internal depth of 200 mm, and comprising 39.2 kg of steel balls having a diameter of less than 10 mm;
the lacquers are prepared in a mixer exhibiting a movement of planetary type sold by Kurabo under the name MS NSB 350N.

### EXAMPLE 1

### Preparation of Compositions Formed of Aluminum Pigments

An amount n_{A} of aluminum powder SP and an amount n_{L} of methyl laurate were introduced into a mill and were subjected to milling at 28 revolutions/min for a time of 8 hours.

At the end of the milling, a slurry was obtained and was sieved through sieves with an opening of 25 μm and then filtered through a plate filter. The paste obtained after filtering was homogenized using a sigma-type blade mixer, with addition of ester in order to adjust the level of ester to 65%.

The test was repeated while modifying the nature and the amount of the aluminum powder SP and the amount of ester, in order to obtain the samples BR03 and BR06 according to table 1.

TABLE 1

| Sample | SP | n_{A} (g) | n_{L} (g) |
|---|---|---|---|
| BR03 | Al-05 | 802 | 2350 |
| BR06 | Al-10 | 892 | 6200 |

The specific procedure of sample BR06 was repeated in order to prepare samples BR07 to BR011 but using, for BR07, the liquid recovered after the sieving/filtering stage of sample BR06 and subsequently using, for the preparation of each of samples BRn (n=8 to 11), the liquid recovered after the sieving/filtering stage of sample BR(n−1).

By way of comparison, the procedure of Example 1 was repeated but using, as milling liquid vehicle, on the one hand, a mixture of white spirit (n_{W}, in g) and of Radiacid (n_{R}, in g) (sample BR01-C, BR05-C) and, on the other hand, a mixture of methyl laurate (n_{L}, in g) and of Radiacid (n_{R}, in g) (sample BR02-C). The specific conditions are shown in the following table 2.

TABLE 2

| Sample | SP | n_{A} (g) | n_{w} (g) | n_{R} (g) | n_{L} (g) |
|---|---|---|---|---|---|
| BR01-C | Al-05 | 802 | 2350 | 124 | — |
| BR02-C | Al-05 | 802 | — | 124 | 2350 |

The particle size of various samples before sieving was determined. 0.32 g of a sample was dispersed in 8 ml of butyl glycol and then the mixture obtained was redispersed in 40 ml of ethanol. After homogenizing using a spatula, the mixture was subjected to ultrasound radiation (60 Hz) for 3 min. The mixture was subsequently characterized using a particle sizer of Malvern Mastersizer 2000 type equipped with the Hydro 2000 S liquid-route measurement module.

The results are shown in the following table 3.

TABLE 3

| Sample | d₁₀ (μm) | D₅₀ (μm) | d₉₀ (μm) |
|---|---|---|---|
| BR01-C | 7.9 | 15.7 | 28.4 |
| BR03 | 6.2 | 12.8 | 24.0 |
| BR02-C | 10.2 | 19.5 | 34.3 |
| BR06 | 15.0 | 24.9 | 40.9 |
| BR07 | 14.8 | 24.4 | 39.7 |
| BR08 | 14.2 | 23.4 | 38.2 |
| BR09 | 15.1 | 24.9 | 40.5 |
| BR10 | 15.5 | 25.5 | 41.6 |
| BR11 | 14.0 | 23.0 | 37.4 |

The comparison of the results for samples BR06 to BR11 according to the invention shows that the use of the recycled milling liquid vehicle does not result in a deterioration in the particle size of the pigments.

The comparison of the results obtained for BR01-C and for BR03 shows that the distribution in the dimensions of particles is substantially equivalent when the “white spirit+Radiacid” mixture of the prior art (sample BR01-C) is replaced by the ester according to the invention (sample BR03).

The comparison of the d₅₀ values obtained for BR03 and for BR02-C shows larger particles when the milling liquid vehicle is a mixture of methyl laurate and Radiacid. It is thus confirmed that, when the milling liquid vehicle is a fatty acid ester, the presence of fatty acid reduces the effectiveness of the milling, in which case the particles undergo a deformation which causes them to grow without undergoing cleavage. The milling then takes place without overmilling, that is to say without generating glitter of low particle size obtained by cleavage of glitter of larger size.

### EXAMPLE 2

### Evaluation of the Covering Power of the Pigments

A lacquer composition is prepared in the following way: 1.5 g of a sample of composition formed of pigments is dispersed in 48.5 g of blue nitrocellulose lacquer and the mixture is homogenized in a mixer. The colored lacquer thus obtained is applied to a sheet of paper.

A blue lacquer was thus prepared, on the one hand with the composition formed of pigments according to the invention BR03 and, on the other hand, with the compositions formed of pigments of the prior art BR01-C and BR02-C.

After application of the respective lacquers to a sheet of paper, the covering power was evaluated by observing, by transparency, the light transmitted through each of the coated sheets of paper. It was thus found that:
the lacquer obtained from the composition formed of pigment BR03 has an identical covering power to that of the lacquer obtained from the composition BR01-C, which confirms that the white spirit/fatty acid mixture can advantageously be replaced as milling liquid vehicle by a fatty acid ester;
the lacquer obtained from BR02-C has a lower covering power than the lacquer obtained from BR03.

This result confirms that the methyl laurate provides a lubricating role during the milling and that the addition of a fatty acid gives a very high overall lubricating power, which tends to limit the effectiveness of the milling and to give particles of larger dimensions. The lacquer composition which comprises the pigments resulting from BR02C consequently has a lower covering power than the lacquer composition which comprises the pigments resulting from BR03.

### Example 3

### Aging Tests

The behavior over time of various compositions formed of pigments according to the invention (BR03, BR06, BR09, BR11) and of the pigment composition BR02-C of the prior art was compared. The comparison was carried out by an accelerated aging test according to the following procedure.

For each composition formed of pigments, two samples were prepared and were stored respectively at a temperature of 4° C., that is to say under conditions under which it is accepted that the composition does not change, and at 50° C., which is supposed to bring about accelerated aging.

During the aging, a portion of each of the samples was withdrawn after 3 months, 6 months and 9 months and, from each portion withdrawn, a blue nitrocellulose lacquer was prepared according to the procedure of example 2.

It is known that, in a composition formed of pigments, the pigments have a tendency to agglomerate over time and that an increase in the degree of agglomeration of the pigments in a composition formed of pigments increases the intensity of the coloring of a paint comprising such a composition formed of pigments, to the detriment of the dyeing strength of the pigments.

The change in the dyeing strength of the pigments was evaluated in the CIELab 1976 calorimetric system expressed in polar coordinates [h, C, L] using the parameter C, which gives information about the saturation of a color. The greater the parameter C, the more saturated and purer the color. The smaller the parameter C, the more the color has deteriorated and tends toward the gray. Thus, if two lacquer compositions having the same blue tint (same parameter h) are compared, the parameter C will be lower for the lacquer in which the pigments are less agglomerated and have a greater dyeing strength.

The values obtained using a Minolta CR300 device are given in the following table 4. ΔC=C_{reference}−Ct, Ct being the value measured after an aging time t.

TABLE 4

| | ΔC (3 months) | ΔC (6 months) | ΔC (9 months) |
|---|---|---|---|
| BR02-C | −0.72 | −0.62 | −0.65 |
| BR03 | −0.10 | 0.00 | −0.16 |
| BR06 | 0.01 | 0.01 | 0.04 |
| BR09 | 0.11 | 0.08 | −0.03 |
| BR11 | −0.03 | −0.11 | 0.24 |

It is thus apparent that, for sample BR03 according to the invention, C varies very little up to 9 months, in contrast to what is observed for sample BR02-C according to the prior art, which comprises oleic acid. Simple visual observation of supports coated with the respective lacquer compositions confirms this result. It is also found that, for the other samples according to the invention (BR06, BR09 and BR11), C virtually does not vary over the 9 months of the aging test.

### EXAMPLE 4

The influence of the pigments on various parameters of a glossy finishing lacquer was evaluated.

A paint composition hereinafter denoted by JB 042, exhibiting “chemical” drying, is prepared in the following way. 20.0 g of a sample of the composition formed of pigments BR03 prepared according to example 1 were mixed with a mixture of constituents comprising 71.67 g of an alkyd urethane resin sold under the name Lixothan UAL 55.55 BT, 1.45 g of combined driers sold under the name Octa Soligen 161/D60, 1.2 g of antiskinning agents sold under the name Exkin 518 and 5.68 g of white spirit. The paint composition thus obtained was homogenized using a mixer.

A reference mixture denoted hereinbelow by JB 041 was prepared in a similar way, the composition formed of pigments BR03 being replaced by the composition formed of pigments of the prior art BR01-C defined in table 2 above. Gloss The metallic paints JB 042 and JB 041 were applied with a wet film thickness of 150 μm to a metal sheet. The gloss of the paint film was evaluated under an angle of 60° and of 85° at different observation times. The results are shown in the following table 5. D+1, D+7 and D+30 denote the time of the observation, that is to say respectively, 1 day, 7 days and 30 days after the deposition of the paint film.

TABLE 5

| | | |
|---|---|---|
| D + 1 | D + 7 | D + 30 |

| | 60° | 85° | 60° | 85° | 60° | 85° |
|---|---|---|---|---|---|---|
| JB 041 | 58.5 | 66.5 | 55.7 | 65.9 | 53.2 | 64.4 |
| JB 042 | 74.5 | 91.1 | 55.5 | 83.5 | 54.4 | 78 |

These results show that the gloss of the film according to the invention is markedly greater than that of the film according to the prior art at D+1. The difference in gloss decreases with time but the gloss nevertheless remains superior for the film according to the invention.

### Accelerated Aging Test

The metallic paints JB 041 and JB 042 were applied with a wet film thickness of 150 μm to a metal sheet. The sheets thus obtained were stored in darkness and their calorimetric coordinates (Lab system) were measured at regular intervals in order to evaluate possible modification to their color (yellowing). The results are given in table 6 below.

TABLE 6

| | D + 1 | | | D + 7 | | | D + 14 | | | D + 30 | | |
|---|---|---|---|---|---|---|---|---|---|---|---|---|
| | L* | a* | b* | L* | a* | b* | L* | a* | b* | L* | a* | b* |
| JB 041 | 78.12 | −0.54 | −1.73 | 78.32 | −0.55 | −1.68 | 78.58 | −0.55 | −1.58 | 78.64 | −0.65 | −1.46 |
| JB 042 | 76 | −0.55 | −1.75 | 76.86 | −0.57 | −1.7 | 76.88 | −0.57 | −1.56 | 77 | −0.67 | −1.39 |

These results show a similar change in the calorimetric coordinates of the paint films, in particular the parameter b*, the change in which toward the positive values indicates a yellowing of the film which increases in the same way for the reference paint JB 041 and for the paint according to the invention JB 042.

### Drying of the Paint Film

The metallic paints JB 041 and JB 042 are applied with a wet film thickness of 100 μm to a glass sheet. A metal ball is held on the wet paint film using a weight of 10 g. A linear translation movement at a constant rate (6 cm/h) is imposed on the ball, which is thus displaced linearly at the surface of the paint film leaving a track, the analysis of which reveals the nature of the drying as a function of the time.

Typically, it is considered that the film is beginning to dry when it no longer closes up again after the ball has passed, that there is surface drying when the film begins to exhibit pick-off, that there is through drying when the pick off disappears and that the drying is complete when the ball no longer leaves any mark on the substrate.

The results obtained are summarized in the following table 7.

TABLE 7

| | JB 041 | JB 042 |
|---|---|---|
| Film no longer closes up again | 20 min | 50 min |
| Beginning of pick off | 30 min | 1 h |
| End of pick off | 4 h 30 | 5 h 10 |
| End of track of the ball* | >11 h | >11 h |

*: the test has a duration of 11 h; it is consequently possible for the track of the ball to be still present after this period of time without this being able to be demonstrated

The initial phase of the drying is extended by 30 min for the metallic paint JB 042 and the time lag taken during the 1st stage of the drying remains stable throughout the various stages. This thus tends to show that the other phases of the drying take place according to similar kinetics to those of the paint of the prior art JB 041.

### Hardness of the Paint Film

The metallic paints JB 041 and JB 042 are applied with a wet film thickness of 150 μm to a metal sheet. The sheets thus produced are subjected to the Persoz pendulum test in order to evaluate their hardness. The pendulum is placed at the surface of the paint film and set in oscillation. The higher the hardness of the film, the weaker the damping of the oscillations and the greater the oscillation time. The hardness can thus be “expressed” in seconds, a longer time indicating a greater hardness. The results are given in table 8 below.

TABLE 8

| | D + l | D + 2 | D + 7 | D + 14 | D + 30 |
|---|---|---|---|---|---|
| JB 041 | 47.3 | 55.7 | 75.3 | 126.3 | 217.3 |
| JB 042 | 47 | 91 | 144.3 | 171 | 209 |

The starting hardness (1 day after application) is identical for the two paint films. Subsequently, the hardness increases more significantly for the film JB 042 according to the invention. However, after 30 days, the two films exhibit a similar hardness.

It can thus be concluded, from the combined tests carried out, that the replacement of the pigments of the prior art by pigments according to the present invention in a paint composition of the alkyd urethane type does not modify the main parameters characteristic of the paint, i.e. gloss, stability, drying time and hardness.

1. A process for the preparation of a composition formed of metal pigments comprising introducing starting metal particles (SP) into a liquid vehicle and subjecting the mixture thus obtained to milling, wherein the liquid vehicle comprises an ester of a fatty acid R′-COOR in which R′ is a saturated or unsaturated aliphatic group having from 9 to 20 carbon atoms and R is an alkyl having from 1 to 8 carbon atoms.

2. The process as claimed in claim 1, wherein the ester R′-COOR is chosen from those in which R′ is a saturated CₙH₂ₙ₊₁ group, n being between 9 and 20.

3. The process as claimed in claim 2, wherein the ester is methyl laurate.

4. The process as claimed in claim 1, wherein the metal particles SP are chosen from particles of aluminum, of copper, of zinc, of tin, of gold, of silver, of alloys of aluminum, copper, zinc, tin, gold, or silver, of stainless steel, or of bronze.

5. The process as claimed in claim 1, wherein the metal particles SP have a mean dimension of 0.1 to 500 μm.

6. The process as claimed in claim 1, wherein the mixture subjected to milling comprises from 1 to 10 kg of liquid vehicle per 1 kg of SP particles.

7. The process as claimed in claim 1, wherein the duration of the milling is between 1 h and 20 h.

8. The process as claimed in claim 1, wherein, at the end of the milling stage, the content of acid ester is adjusted to a value of between 30 and 90% by weight of the total weight.

9. A composition formed of metal pigments comprising metal particles FP and a liquid, wherein the liquid is an ester of a fatty acid R′-COOR in which R′ is a saturated or unsaturated aliphatic group having from 9 to 20 carbon atoms and R is an alkyl having from 1 to 8 carbon atoms.

10. The composition as claimed in claim 9, wherein R is a methyl and R′ is a saturated alkyl group having from 9 to 17 carbon atoms.

11. The composition as claimed in claim 1, wherein the ester content is between 30 and 90% by weight.

12. The composition as claimed in claim 9, wherein the metal particles FP are anisotropic particles having mean dimensions of less than or equal to 500 μm and a form factor, which is the ratio of the mean thickness to the mean transverse diameter, of between 1/5 and 1/1000.

13. The composition as claimed in claim 12, wherein the particles are of the glitter type with a mean transverse diameter of less than or equal to 500 μm and a mean thickness of less than or equal to 3 μm.

14. The composition as claimed in claim 9, wherein the ester is methyl laurate and the metal particles are aluminum glitter.

15. A paint composition comprising a composition formed of metal pigments as claimed in claim 9.

16. An ink composition comprising a composition formed of metal pigments as claimed in claim 9.

17. A composition formed of plastic with a metallic appearance comprising a composition formed of metal pigments as claimed in claim 9.

18. A cosmetic composition with a metallic appearance comprising a composition formed of metal pigments as claimed in claim 9.
